(19) United States
(12) Patent Application Publication
(10) Pub. No.: US 2015/0352099 A1
(43) Pub. Date: Dec. 10, 2015

(54) Compositions and Methods of Reducing Sedation

(71) Applicants: Mentinova Inc., New Brunswick, NJ (US); Rutgers, The State University of New Jersey, New Brunswick, NJ (US)

(72) Inventors: Steven Braithwaite, Redwood City, CA (US); Michael Voronkov, Pennington, NJ (US); M. Maral Mouradian, Princeton, NJ (US)

(73) Assignees: Mentinova Inc., New Brunswick, NJ (US); Rutgers, The State University of New Jersey, New Brunswick, NJ (US)

(21) Appl. No.: 14/730,717

(22) Filed: Jun. 4, 2015

Related U.S. Application Data
(60) Provisional application No. 62/007,425, filed on Jun. 4, 2014.

(51) Int. Cl. A61K 31/485; A61K 31/277; A61K 31/198

(52) U.S. Cl. CPC A61K 31/485; A61K 31/198; A61K 31/277

(57) ABSTRACT

Methods and compositions useful in reducing drug-induced sedation in subjects.

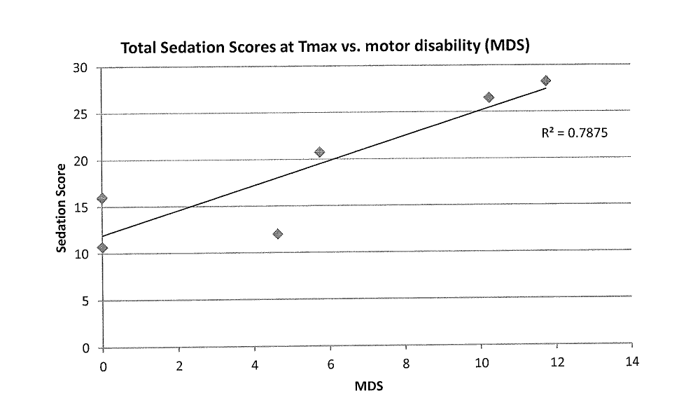

### RELATED APPLICATIONS

The present application claims priority to U.S. Provisional Patent Application No. 62/007,425, filed Jun. 4, 2014, the disclosure of which is incorporated by reference herein in its entirety.

### FIELD OF THE INVENTION

The present invention relates to compositions and methods useful in reducing sedation associated with opioid receptor modulation.

### BACKGROUND

Opioids are commonly used in pain management. Opium, derived from poppy plants is an opioid, as are natural derivatives of opium (opiates), including morphine, methadone, and heroin. The term “opioids” represents a broad class of drugs that includes not only opium and opiates, but also synthetic drugs with the same pharmacological effect as opium including meperidine, fentanyl, alfentanil, sufentanil, and remifentanil. Opioids are powerful analgesics, but opioids are also powerful sedatives. The amount of opioid administered to a patient and the level of pain relief a patient receives from an opioid is currently limited by the patient's level of sedation induced by the opioid. There is a need for compositions and methods for administering an opioid that reduce or substantially eliminate the sedative effects of the opioid.

### SUMMARY OF THE INVENTION

The present invention relates to a combination of opioid agents or related compounds with a dopaminergic agent as a means of reducing sedation caused by opiate or related compounds in subjects and uses thereof.

The present invention further relates to methods of mitigating opiate adverse effects such as sedation when opiates are used in the following, but not limited to, conditions: pain management, palliative care, anesthesiology (e.g. postoperatively), skin disorders (e.g. pruritus), addictions (detox or management), etc.

### BRIEF DESCRIPTION OF THE DRAWINGS

The foregoing summary, as well as the following detailed description of the invention, will be better understood when read in conjunction with the appended drawings. The FIGURE is a graph showing total sedation scores as a function of motor disability in non-human primates treated with nalbuphine HCl monotherapy.

### DETAILED DESCRIPTION OF INVENTION

The present invention relates to the administration of a dopaminergic agent (e.g., dopamine agonists, L-DOPA, MAO-B inhibitors, and COMT inhibitors) with opioids (e.g. fentanyl, hydrocodone, hydromorphine, morphine, oxycodone, diacetylmorphine, methadone, nalbuphine, pentazocine, butorphanol, etc. and salts thereof, as described in more detail below) or related compounds.

The administration can be contemporaneous (e.g., co-administration) or sequential. The dopaminergic agent can be administered in an amount sufficient to suppress an opiates' adverse effects, such as sedation.

The present invention offers a novel method of treatment with the advantage of reducing or eliminating the incidence of adverse effects such as sedation. In certain embodiments, this therapeutic provides superior quality of care and allows a wider range of patients who otherwise may not be suitable for opioid therapy to benefit from this analgesic.

Unexpectedly, it is now found that there is a correlation between brain dopamine levels in primates, as inferred by the severity of Parkinsonian signs, and sedation produced by an opiate. More specifically, as shown in the FIGURE, it is now found that parkinsonian motor disability scores (MDS) correlate with sedation scores when nalbuphine is administered in doses of 0.016 mg/kg-0.50 mg/kg in primates. In other words, severity of dopamine deficiency is a predictor of the sedation severity for the same dose of nalbuphine given to different animals.

Surprisingly, it is now found that administration of a dopaminergic agent such as L-DOPA to boost brain dopamine levels attenuates and in some embodiments completely attenuates, any opioid sedation in the same cohort of animals. With respect to nalbuphine, in subjects with low levels of dopamine it is very surprising that co-administration of L-DOPA with nalbuphine suppresses such a common adverse effect such as sedation. This could not be anticipated, as generally L-DOPA use is not known to increase alertness, and, in some cases, administration of L-DOPA or dopamine agonists could be associated with adverse effects such as sedation and somnolence or even sudden onset of sleep without warning.

Preferably the opioid provides the same level of analgesia to a patient when administered in combination with a dopaminergic agent as the same amount of opioid provides when administered alone, however the sedative effects of the opioid are reduced or substantially eliminated when the opioid is administered in combination with the dopaminergic agent. In some embodiments administration of a dopaminergic agent and an opioid does not substantially affect one or more of the pharmacokinetic parameters (e.g. Tₘₐₓ, Cₘₐₓ, AUC) of the dopaminergic agent and/or the opioid compared to when each is administered individually. In some embodiments administration of a dopaminergic agent and an opioid agent does not substantially affect the maximum plasma concentration (Cmax) of the opioid as compared to administration of the opioid alone. In an embodiment where an opioid (e.g. nalbuphine) is administered subcutaneously at a dosage of 0.25 mg/kg, the average Cmax is about 49 ng/mL. In an embodiment where an opioid (e.g. nalbuphine) is administered subcutaneously at a dosage of 0.25 mg/kg and a dopaminergic agent (e.g. L-DOPA) is administered subcutaneously at a dosage of 25 mg/kg, the average Cmax of opioid is about 55 ng/mL. In some embodiments, administration of a dopaminergic agent and opioid does not affect the area under the curve of plasma concentration versus time (AUC) of the opioid as compared to administration of the opioid alone. In an embodiment where an opioid (e.g. nalbuphine) is administered subcutaneously at a dosage of 0.25 mg/kg, the average AUC is about 96 ng*hr/mL. In an embodiment where an opioid (e.g. nalbuphine) is administered subcutaneously at a dosage of 0.25 mg/kg and a dopaminergic agent (e.g. L-DOPA) is administered subcutaneously at a dosage of 25 mg/kg, the average AUC of opioid is about 89 ng*hr/mL. In some embodiments, administration of a dopaminergic agent and an opioid agent does not affect the amount of time the opioid is present at the maximum concentration in blood serum (Tmax) as compared to administration of the opioid alone. In an embodiment where an opioid (e.g. nalbuphine) is administered subcutaneously at a dosage of 0.25 mg/kg, the average Tmax is about 37 min. In an embodiment where an opioid (e.g. nalbuphine) is administered subcutaneously at a dosage of 0.25 mg/kg and a dopaminergic agent (e.g. L-DOPA) is administered subcutaneously at a dosage of 25 mg/kg, the average Tmax of opioid is about 32 min.

The present invention encompasses pharmaceutical compositions and methods to reduce or prevent sedation or one or more symptoms of sedation by co-administering a dopaminergic agent when an opioid agent is used in pain management, anesthesia (e.g. postoperatively), skin disorders (e.g. pruritus), addictions (detox or management) and other conditions in a subject. Opiate adverse effects include decreased attentiveness, diminished reactivity, drowsiness, and combinations thereof.

Compositions for Use with the Present Invention

In one embodiment, the present invention is a method of reducing, preventing, or substantially eliminating opiate adverse effects such as sedation by administering separately or simultaneously as a composition comprising a (1.) dopaminergic agent and (2.) an opioid agent. In another embodiment, the dosage form includes a dopaminergic agent in an amount or weight ratio relative to an opioid agent sufficient to provide the benefit of the opioid, but the dopaminergic agent is present in sufficient amount to diminish one or more symptoms of decreased attentiveness, diminished reactivity, drowsiness in a patient compared to the same amount of opioid agent alone. In one embodiment the method includes administering a dopaminergic agent and an opioid agent to a patient. In one embodiment the patient is an animal. In one embodiment the animal is a human. In one embodiment the patient is an animal (e.g. a human) in need of treatment with an opioid.

1. Dopaminergic Agents

Exemplary dopaminergic agents for use in certain embodiments of the present invention include one or more of: (a) a dopamine agonist, (b) L-DOPA, (c) an MAO inhibitor, (d) a COMT inhibitor, or (e) a dopamine re-uptake inhibitor. In certain embodiments, the dopaminergic agent is administered in an amount sufficient to reduce or prevent sedation caused by opioid administration.

(a) Dopamine Agonists

Dopamine agonists useful in the present invention include, but are not limited to, pramipexole, ropinirole, rotigotine, apomorphine, piribedil, cabergoline, lisuride, and derivatives, prodrugs, esters, and salts thereof.

In some embodiments a dopamine agonist is administered in a dosage range of about 0.5 mg to about 4 mg, about 1 mg to about 3 mg, about 0.1 mg to about 1 mg, about 0.5 mg to about 2.5 mg, about 2.5 mg to about 4.5 mg, about 4.5 mg to about 7 mg, about 7 mg to about 10 mg, about 0.5 mg to about 10 mg, or about 2 mg to about 8 mg. In some embodiments a dopamine agonist is administered at a dose of about 0.1 mg, about 0.125 mg, about 0.375, about 0.5 mg, about 0.75, about 1 mg, about 1.5 mg, about 2 mg, about 2.5 mg, about 3 mg, about 3.5 mg, about 4 mg, about 4.5 mg, about 5 mg, about 6 mg, about 7 mg, about 8 mg, about 9 mg, or about 10 mg.

(b) L-DOPA

In some embodiments L-DOPA is administered in a dosage range of about 10 mg to about 200 mg, about 30 mg to about 90 mg, about 40 mg to about 80 mg, about 50 mg to about 70 mg, about 20 mg to about 40 mg, about 30 mg to about 50 mg, about 40 mg to about 60 mg, about 60 mg to about 80 mg, about 70 mg to about 90 mg, about 80 mg to about 100 mg, about 25 mg to about 175 mg, about 50 mg to about 150 mg, about 75 mg to about 125 mg, about 10 mg to about 25 mg, about 100 mg to about 125 mg, about 125 mg to about 150 mg, about 150 mg to about 175 mg, or about 175 mg to about 200 mg. In some embodiments L-DOPA is administered in a dosage of about 10 mg, about 15 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, or about 200 mg.

(c) MAO Inhibitors

Monoamine oxidase inhibitors (MAO inhibitors) useful in the present invention include, but are not limited to, isocarboxazid, nialamide, phenelzine, hydracarbazine, tranylcypromie, moclobemide, pirlindole, toloxatone, rasagiline, selegiline, linezolid, and derivatives, prodrugs, esters, and salts thereof. In some embodiments the MAO inhibitor is an MAO B inhibitor, while in other embodiments the MAO inhibitor is an MAO A inhibitor.

In some embodiments an MAO inhibitor is administered in a dosage range of about 0.1 mg to about 200 mg, about 0.1 mg to about 100 mg, about 0.1 mg to about 25 mg, about 0.1 mg to about 10 mg, about 0.1 mg to about 5 mg, about 0.5 mg to about 5 mg, about 0.5 mg to about 2.5 mg, about 1 mg to about 2.5 mg, about 1 mg to about 5 mg, about 1 mg to about 7.5 mg, about 1 mg to about 10 mg, about 10 mg to about 200 mg, about 25 mg to about 175 mg, about 50 mg to about 150 mg, about 75 mg to about 125 mg, about 10 mg to about 25 mg, about 25 mg to about 50 mg, about 50 mg to about 75 mg, about 75 mg to about 100 mg, about 100 mg to about 125 mg, about 125 mg to about 150 mg, about 150 mg to about 175 mg, or about 175 mg to about 200 mg. In some embodiments an MAO inhibitor is administered at a dose of about 0.1 mg, about 0.25 mg, about 0.5 mg, about 0.75 mg, about 1 mg, about 1.25 mg, about 1.5 mg, about 1.75 mg, about 2 mg, about 2.5 mg, about 3 mg, about 3.5 mg, about 4 mg, about 4.5 mg, about 5 mg, about 6 mg, about 7 mg, about 8 mg, about 9 mg, 10 mg, about 15 mg, about 20 mg, about 25 mg, about 50, about 75 mg, about 100 mg, about 125 mg, about 150 mg, about 175 mg, or about 200 mg.

(d) COMT Inhibitors

Catechol-O-methyl transferase inhibitors (COMT inhibitors) useful in the present invention include, but are not limited to, entacapone, tolcapone, nitecapone, opicapone, and derivatives, prodrugs, esters, and salts thereof.

In some embodiments a COMT inhibitor is administered in a dosage range of about 10 mg to about 200 mg, about 25 mg to about 175 mg, about 50 mg to about 150 mg, about 75 mg to about 125 mg, about 10 mg to about 25 mg, about 25 mg to about 50 mg, about 50 mg to about 75 mg, about 75 mg to about 100 mg, about 100 mg to about 125 mg, about 125 mg to about 150 mg, about 150 mg to about 175 mg, or about 175 mg to about 200 mg. In some embodiments a COMT inhibitor is administered at a dose of about 10 mg, about 15 mg, about 20 mg, about 25 mg, about 50, about 75 mg, about 100 mg, about 125 mg, about 150 mg, about 175 mg, or about 200 mg.

(e) Dopamine Re-Uptake Inhibitors

Dopamine re-uptake inhibitors useful in the present invention include, but are not limited to, amineptine, bromantane, dexmethylphenidate, difemetorex, fencamfamine, lefetamine, levophacetoperane, medifoxamine, mesocarb, methylphenidate, nomifensine, pipradrol, prolintane, pyrovalerone, adrafinil, armodafinil, bupropion, mazindol, modafinil, nefazodone, sertraline, sibutramine, and derivatives, prodrugs, esters, and salts thereof.

In some embodiments a dopamine re-uptake inhibitor is administered in a dosage range of about 10 mg to about 200 mg, about 25 mg to about 175 mg, about 50 mg to about 150 mg, about 75 mg to about 125 mg, about 10 mg to about 25 mg, about 25 mg to about 50 mg, about 50 mg to about 75 mg, about 75 mg to about 100 mg, about 100 mg to about 125 mg, about 125 mg to about 150 mg, about 150 mg to about 175 mg, or about 175 mg to about 200 mg. In some embodiments a dopamine re-uptake inhibitor is administered at a dose of about 10 mg, about 15 mg, about 20 mg, about 25 mg, about 50, about 75 mg, about 100 mg, about 125 mg, about 150 mg, about 175 mg, or about 200 mg.

2. Opioid Agents

Opioid agents (opioid receptor modulators) useful in the present invention include, but are not limited to, one or more of a mu, delta and kappa receptor ligand.

In certain embodiments, the opioid agent or agents is one or more of: fentanyl, hydrocodone, hydromorphine, morphine, oxycodone, diacetylmorphine, methadone, nalbuphine, pentazocine, butorphanol, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, and tramodol or derivative, prodrug, or pharmaceutically acceptable salt thereof.

In some embodiments, the opioid agent is nalbuphine HCl.

In some embodiments the opioid agent is administered in a dosage range of about 0.01 mg to about 100 mg, about 0.01 mg to about 50 mg, about 0.01 mg to about 25 mg, about 0.01 mg to about 10 mg, about 0.01 mg to about 1 mg, about 0.025 mg to about 0.5 mg, about 0.025 mg to about 0.25 mg, about 0.05 mg to about 0.1 mg, about 1 mg to about 250 mg, about 10 mg to about 100 mg, about 20 mg to about 80 mg, about 25 mg to about 75 mg, about 40 mg to about 60 mg, about 5 mg to about 25 mg, about 25 mg to about 50 mg, about 50 mg to about 75 mg, about 75 mg to about 100 mg, about 100 mg to about 125 mg, or about 125 mg to about 150 mg. In some embodiments the opioid agent is administered at a dose of about 0.01 mg, about 0.025 mg, about 0.05 mg, about 0.075 mg, about 0.1 mg, about 0.15 mg, about 0.2 mg, about 0.25 mg, about 0.3 mg, about 0.35 mg, about 0.4 mg, about 0.45 mg, about 0.5 mg, about 0.6 mg, about 0.7 mg, about 0.75 mg, about 0.8 mg, about 0.9 mg, about 1 mg, about 2 mg, about 2.5 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In some embodiments, one or more opioid agents are administered in an amount sufficient to cause sedation or other adverse effect including such effects described herein.

Non-limiting examples of compositions comprising a dopamine agonist and an opioid agent in accordance with the invention are provided in Table 1.

TABLE 1

| Example | Dopamine | | | |
|---|---|---|---|---|
| No. | Agonist | Dosage | Opioid Agent | Dosage |
| 1 | pramipexole | about 0.125 mg | nalbuphine | about 5 mg to about 25 mg |
| 2 | pramipexole | about 0.125 mg | nalbuphine | about 5 mg to about |
| | | | | 150 mg |
| 3 | pramipexole | about 1.5 mg | nalbuphine | about 5 mg to about 25 mg |
| 4 | pramipexole | about 1.5 mg | nalbuphine | about 5 mg to about |
| | | | | 150 mg |
| 5 | pramipexole | about 0.1 mg to | nalbuphine | about 5 mg to about |
| | | about 4.5 mg | | 150 mg |
| 6 | pramipexole | about 0.1 mg to | pentazocine | about 5 mg to about |
| | | about 4.5 mg | | 150 mg |
| 7 | pramipexole | about 0.1 mg to | butorphanol | about 5 mg to about |
| | | about 4.5 mg | | 150 mg |
| 8 | pramipexole | about 0.1 mg to | fentanyl | about 0.01 mg to about |
| | | about 4.5 mg | | 25 mg |
| 9 | pramipexole | about 0.1 mg to | hydrocodone | about 0.01 mg to about |
| | | about 4.5 mg | | 25 mg |
| 10 | pramipexole | about 0.1 mg to | hydromorphone | about 0.01 mg to about |
| | | about 4.5 mg | | 25 mg |
| 11 | pramipexole | about 0.1 mg to | morphine | about 0.01 mg to about |
| | | about 4.5 mg | | 50 mg |
| 12 | pramipexole | about 0.1 mg to | oxycodone | about 0.01 mg to about |
| | | about 4.5 mg | | 50 mg |
| 13 | pramipexole | about 0.1 mg to | meperidine | about 1 mg to about |
| | | about 4.5 mg | | 250 mg |
| 14 | pramipexole | about 0.1 mg to | codeine | about 1 mg to about |
| | | about 4.5 mg | | 250 mg |
| 15 | ropinirole | about 0.1 mg to | nalbuphine | about 5 mg to about |
| | | about 25 mg | | 150 mg |
| 16 | ropinirole | about 0.1 mg to | pentazocine | about 5 mg to about |
| | | about 25 mg | | 150 mg |
| 17 | ropinirole | about 0.1 mg to | butorphanol | about 5 mg to about |
| | | about 25 mg | | 150 mg |
| 18 | ropinirole | about 0.1 mg to | fentanyl | about 0.01 mg to about |
| | | about 25 mg | | 25 mg |
| 19 | ropinirole | about 0.1 mg to | hydrocodone | about 0.01 mg to about |
| | | about 25 mg | | 25 mg |
| 20 | ropinirole | about 0.1 mg to | hydromorphone | about 0.01 mg to about |
| | | about 25 mg | | 25 mg |
| 21 | ropinirole | about 0.1 mg to | morphine | about 0.01 mg to about |
| | | about 25 mg | | 50 mg |
| 22 | ropinirole | about 0.1 mg to | oxycodone | about 0.01 mg to about |
| | | about 25 mg | | 50 mg |
| 23 | ropinirole | about 0.1 mg to | meperidine | about 1 mg to about |
| | | about 25 mg | | 250 mg |
| 24 | ropinirole | about 0.1 mg to | codeine | about 1 mg to about |
| | | about 25 mg | | 250 mg |
| 25 | rotigotine | about 1 mg to about | nalbuphine | about 5 mg to about |
| | | 10 mg | | 150 mg |
| 26 | rotigotine | about 1 mg to about | pentazocine | about 5 mg to about |
| | | 10 mg | | 150 mg |
| 27 | rotigotine | about 1 mg to about | butorphanol | about 5 mg to about |
| | | 10 mg | | 150 mg |
| 28 | rotigotine | about 1 mg to about | fentanyl | about 0.01 mg to about |
| | | 10 mg | | 25 mg |
| 29 | rotigotine | about 1 mg to about | hydrocodone | about 0.01 mg to about |
| | | 10 mg | | 25 mg |
| 30 | rotigotine | about 1 mg to about | hydromorphone | about 0.01 mg to about |
| | | 10 mg | | 25 mg |
| 31 | rotigotine | about 1 mg to about | morphine | about 0.01 mg to about |
| | | 10 mg | | 50 mg |
| 32 | rotigotine | about 1 mg to about | oxycodone | about 0.01 mg to about |
| | | 10 mg | | 50 mg |
| 33 | rotigotine | about 1 mg to about | meperidine | about 1 mg to about |
| | | 10 mg | | 250 mg |
| 34 | rotigotine | about 1 mg to about | codeine | about 1 mg to about |
| | | 10 mg | | 250 mg |
| 35 | apomorphine | about 1 mg to about | nalbuphine | about 5 mg to about |
| | | 10 mg | | 150 mg |
| 36 | apomorphine | about 1 mg to about | pentazocine | about 5 mg to about |
| | | 10 mg | | 150 mg |
| 37 | apomorphine | about 1 mg to about | butorphanol | about 5 mg to about |
| | | 10 mg | | 150 mg |
| 38 | apomorphine | about 1 mg to about | fentanyl | about 0.01 mg to about |
| | | 10 mg | | 25 mg |
| 39 | apomorphine | about 1 mg to about | hydrocodone | about 0.01 mg to about |
| | | 10 mg | | 25 mg |
| 40 | apomorphine | about 1 mg to about | hydromorphone | about 0.01 mg to about |
| | | 10 mg | | 25 mg |
| 41 | apomorphine | about 1 mg to about | morphine | about 0.01 mg to about |
| | | 10 mg | | 50 mg |
| 42 | apomorphine | about 1 mg to about | oxycodone | about 0.01 mg to about |
| | | 10 mg | | 50 mg |
| 43 | apomorphine | about 1 mg to about | meperidine | about 1 mg to about |
| | | 10 mg | | 250 mg |
| 44 | apomorphine | about 1 mg to about | codeine | about 1 mg to about |
| | | 10 mg | | 250 mg |
| 45 | piribedil | about 25 mg to about | nalbuphine | about 5 mg to about |
| | | 75 mg | | 150 mg |
| 46 | piribedil | about 25 mg to about | pentazocine | about 5 mg to about |
| | | 75 mg | | 150 mg |
| 47 | piribedil | about 25 mg to about | butorphanol | about 5 mg to about |
| | | 75 mg | | 150 mg |
| 48 | piribedil | about 25 mg to about | fentanyl | about 0.01 mg to about |
| | | 75 mg | | 25 mg |
| 49 | piribedil | about 25 mg to about | hydrocodone | about 0.01 mg to about |
| | | 75 mg | | 25 mg |
| 50 | piribedil | about 25 mg to about | hydromorphone | about 0.01 mg to about |
| | | 75 mg | | 25 mg |
| 51 | piribedil | about 25 mg to about | morphine | about 0.01 mg to about |
| | | 75 mg | | 50 mg |
| 52 | piribedil | about 25 mg to about | oxycodone | about 0.01 mg to about |
| | | 75 mg | | 50 mg |
| 53 | piribedil | about 25 mg to about | meperidine | about 1 mg to about |
| | | 75 mg | | 250 mg |
| 54 | piribedil | about 25 mg to about | codeine | about 1 mg to about |
| | | 75 mg | | 250 mg |
| 55 | cabergoline | about 0.1 mg to | nalbuphine | about 5 mg to about |
| | | about 4.5 mg | | 150 mg |
| 56 | cabergoline | about 0.1 mg to | pentazocine | about 5 mg to about |
| | | about 4.5 mg | | 150 mg |
| 57 | cabergoline | about 0.1 mg to | butorphanol | about 5 mg to about |
| | | about 4.5 mg | | 150 mg |
| 58 | cabergoline | about 0.1 mg to | fentanyl | about 0.01 mg to about |
| | | about 4.5 mg | | 25 mg |
| 59 | cabergoline | about 0.1 mg to | hydrocodone | about 0.01 mg to about |
| | | about 4.5 mg | | 25 mg |
| 60 | cabergoline | about 0.1 mg to | hydromorphone | about 0.01 mg to about |
| | | about 4.5 mg | | 25 mg |
| 61 | cabergoline | about 0.1 mg to | morphine | about 0.01 mg to about |
| | | about 4.5 mg | | 50 mg |
| 62 | cabergoline | about 0.1 mg to | oxycodone | about 0.01 mg to about |
| | | about 4.5 mg | | 50 mg |
| 63 | cabergoline | about 0.1 mg to | meperidine | about 1 mg to about |
| | | about 4.5 mg | | 250 mg |
| 64 | cabergoline | about 0.1 mg to | codeine | about 1 mg to about |
| | | about 4.5 mg | | 250 mg |
| 65 | lisuride | about 0.1 mg to | nalbuphine | about 5 mg to about |
| | | about 4.5 mg | | 150 mg |
| 66 | lisuride | about 0.1 mg to | pentazocine | about 5 mg to about |
| | | about 4.5 mg | | 150 mg |
| 67 | lisuride | about 0.1 mg to | butorphanol | about 5 mg to about |
| | | about 4.5 mg | | 150 mg |
| 68 | lisuride | about 0.1 mg to | fentanyl | about 0.01 mg to about |
| | | about 4.5 mg | | 25 mg |
| 69 | lisuride | about 0.1 mg to | hydrocodone | about 0.01 mg to about |
| | | about 4.5 mg | | 25 mg |
| 70 | lisuride | about 0.1 mg to | hydromorphone | about 0.01 mg to about |
| | | about 4.5 mg | | 25 mg |
| 71 | lisuride | about 0.1 mg to | morphine | about 0.01 mg to about |
| | | about 4.5 mg | | 50 mg |
| 72 | lisuride | about 0.1 mg to | oxycodone | about 0.01 mg to about |
| | | about 4.5 mg | | 50 mg |
| 73 | lisuride | about 0.1 mg to | meperidine | about 1 mg to about |
| | | about 4.5 mg | | 250 mg |
| 74 | lisuride | about 0.1 mg to | codeine | about 1 mg to about |
| | | about 4.5 mg | | 250 mg |

Non-limiting examples of compositions comprising a COMT inhibitor and an opioid agent in accordance with the invention are provided in Table 2.

TABLE 2

| Example | | | | |
|---|---|---|---|---|
| No. | COMT Inhibitor | Dosage | Opioid Agent | Dosage |
| 75 | entacapone | about 10 mg to about | nalbuphine | about 5 mg to about |
| | | 200 mg | | 150 mg |
| 76 | entacapone | about 10 mg to about | nalbuphine | about 5 mg to about 25 mg |
| | | 200 mg | | |
| 77 | entacapone | about 100 mg to | nalbuphine | about 5 mg to about |
| | | about 200 mg | | 150 mg |
| 78 | entacapone | about 100 mg to | nalbuphine | about 5 mg to about 25 mg |
| | | about 200 mg | | |
| 79 | entacapone | about 200 mg | nalbuphine | about 5 mg to about 25 mg |
| 80 | entacapone | about 200 mg | nalbuphine | about 5 mg to about |
| | | | | 150 mg |
| 81 | entacapone | about 10 mg to about | pentazocine | about 5 mg to about |
| | | 200 mg | | 150 mg |
| 82 | entacapone | about 10 mg to about | butorphanol | about 5 mg to about |
| | | 200 mg | | 150 mg |
| 83 | entacapone | about 10 mg to about | fentanyl | about 0.01 mg to about |
| | | 200 mg | | 25 mg |
| 84 | entacapone | about 10 mg to about | hydrocodone | about 0.01 mg to about |
| | | 200 mg | | 25 mg |
| 85 | entacapone | about 10 mg to about | hydromorphone | about 0.01 mg to about |
| | | 200 mg | | 25 mg |
| 86 | entacapone | about 10 mg to about | morphine | about 0.01 mg to about |
| | | 200 mg | | 50 mg |
| 87 | entacapone | about 10 mg to about | oxycodone | about 0.01 mg to about |
| | | 200 mg | | 50 mg |
| 88 | entacapone | about 10 mg to about | meperidine | about 1 mg to about |
| | | 200 mg | | 250 mg |
| 89 | entacapone | about 10 mg to about | codeine | about 1 mg to about |
| | | 200 mg | | 250 mg |
| 90 | tolcapone | about 10 mg to about | pentazocine | about 5 mg to about |
| | | 200 mg | | 150 mg |
| 91 | tolcapone | about 10 mg to about | butorphanol | about 5 mg to about |
| | | 200 mg | | 150 mg |
| 92 | tolcapone | about 10 mg to about | nalbuphine | about 5 mg to about |
| | | 200 mg | | 150 mg |
| 93 | tolcapone | about 10 mg to about | fentanyl | about 0.01 mg to about |
| | | 200 mg | | 25 mg |
| 94 | tolcapone | about 10 mg to about | hydrocodone | about 0.01 mg to about |
| | | 200 mg | | 25 mg |
| 95 | tolcapone | about 10 mg to about | hydromorphone | about 0.01 mg to about |
| | | 200 mg | | 25 mg |
| 96 | tolcapone | about 10 mg to about | morphine | about 0.01 mg to about |
| | | 200 mg | | 50 mg |
| 97 | tolcapone | about 10 mg to about | oxycodone | about 0.01 mg to about |
| | | 200 mg | | 50 mg |
| 98 | tolcapone | about 10 mg to about | meperidine | about 1 mg to about |
| | | 200 mg | | 250 mg |
| 99 | tolcapone | about 10 mg to about | codeine | about 1 mg to about |
| | | 200 mg | | 250 mg |
| 100 | nitecapone | about 10 mg to about | pentazocine | about 5 mg to about |
| | | 200 mg | | 150 mg |
| 101 | nitecapone | about 10 mg to about | butorphanol | about 5 mg to about |
| | | 200 mg | | 150 mg |
| 102 | nitecapone | about 10 mg to about | nalbuphine | about 5 mg to about |
| | | 200 mg | | 150 mg |
| 103 | nitecapone | about 10 mg to about | fentanyl | about 0.01 mg to about |
| | | 200 mg | | 25 mg |
| 104 | nitecapone | about 10 mg to about | hydrocodone | about 0.01 mg to about |
| | | 200 mg | | 25 mg |
| 105 | nitecapone | about 10 mg to about | hydromorphone | about 0.01 mg to about |
| | | 200 mg | | 25 mg |
| 106 | nitecapone | about 10 mg to about | morphine | about 0.01 mg to about |
| | | 200 mg | | 50 mg |
| 107 | nitecapone | about 10 mg to about | oxycodone | about 0.01 mg to about |
| | | 200 mg | | 50 mg |
| 108 | nitecapone | about 10 mg to about | meperidine | about 1 mg to about |
| | | 200 mg | | 250 mg |
| 109 | nitecapone | about 10 mg to about | codeine | about 1 mg to about |
| | | 200 mg | | 250 mg |
| 110 | opicapone | about 10 mg to about | pentazocine | about 5 mg to about |
| | | 200 mg | | 150 mg |
| 111 | opicapone | about 10 mg to about | butorphanol | about 5 mg to about |
| | | 200 mg | | 150 mg |
| 112 | opicapone | about 10 mg to about | nalbuphine | about 5 mg to about |
| | | 200 mg | | 150 mg |
| 113 | opicapone | about 10 mg to about | fentanyl | about 0.01 mg to about |
| | | 200 mg | | 25 mg |
| 114 | opicapone | about 10 mg to about | hydrocodone | about 0.01 mg to about |
| | | 200 mg | | 25 mg |
| 115 | opicapone | about 10 mg to about | hydromorphone | about 0.01 mg to about |
| | | 200 mg | | 25 mg |
| 116 | opicapone | about 10 mg to about | morphine | about 0.01 mg to about |
| | | 200 mg | | 50 mg |
| 117 | opicapone | about 10 mg to about | oxycodone | about 0.01 mg to about |
| | | 200 mg | | 50 mg |
| 118 | opicapone | about 10 mg to about | meperidine | about 1 mg to about |
| | | 200 mg | | 250 mg |
| 119 | opicapone | about 10 mg to about | codeine | about 1 mg to about |
| | | 200 mg | | 250 mg |

Non-limiting examples of compositions comprising L-DOPA and an opioid agent in accordance with the invention are provided in Table 3.

TABLE 3

| Example | | | | |
|---|---|---|---|---|
| No. | L-DOPA | Dosage | Opioid Agent | Dosage |
| 120 | L-DOPA | about 10 mg to about | nalbuphine | about 5 mg to about |
| | | 200 mg | | 150 mg |
| 121 | L-DOPA | about 10 mg to about | nalbuphine | about 5 mg to about 25 mg |
| | | 200 mg | | |
| 122 | L-DOPA | about 25 mg to about | nalbuphine | about 5 mg to about |
| | | 75 mg | | 150 mg |
| 123 | L-DOPA | about 25 mg to about | nalbuphine | about 5 mg to about 25 mg |
| | | 75 mg | | |
| 124 | L-DOPA | about 50 mg | nalbuphine | about 5 mg to about 25 mg |
| 125 | L-DOPA | about 50 mg | nalbuphine | about 5 mg to about |
| | | | | 150 mg |
| 126 | L-DOPA | about 10 mg to about | pentazocine | about 5 mg to about |
| | | 200 mg | | 150 mg |
| 127 | L-DOPA | about 10 mg to about | pentazocine | about 5 mg to about 25 mg |
| | | 200 mg | | |
| 128 | L-DOPA | about 25 mg to about | pentazocine | about 5 mg to about |
| | | 75 mg | | 150 mg |
| 129 | L-DOPA | about 25 mg to about | pentazocine | about 5 mg to about 25 mg |
| | | 75 mg | | |
| 130 | L-DOPA | about 50 mg | pentazocine | about 5 mg to about 25 mg |
| 131 | L-DOPA | about 50 mg | pentazocine | about 5 mg to about |
| | | | | 150 mg |
| 132 | L-DOPA | about 10 mg to about | butorphanol | about 5 mg to about |
| | | 200 mg | | 150 mg |
| 133 | L-DOPA | about 10 mg to about | butorphanol | about 5 mg to about 25 mg |
| | | 200 mg | | |
| 134 | L-DOPA | about 25 mg to about | butorphanol | about 5 mg to about |
| | | 75 mg | | 150 mg |
| 135 | L-DOPA | about 25 mg to about | butorphanol | about 5 mg to about 25 mg |
| | | 75 mg | | |
| 136 | L-DOPA | about 50 mg | butorphanol | about 5 mg to about 25 mg |
| 137 | L-DOPA | about 50 mg | butorphanol | about 5 mg to about |
| | | | | 150 mg |
| 138 | L-DOPA | about 10 mg to about | fentanyl | about 0.01 mg to about |
| | | 200 mg | | 25 mg |
| 139 | L-DOPA | about 10 mg to about | hydrocodone | about 0.01 mg to about |
| | | 200 mg | | 25 mg |
| 140 | L-DOPA | about 10 mg to about | hydromorphone | about 0.01 mg to about |
| | | 200 mg | | 25 mg |
| 141 | L-DOPA | about 10 mg to about | morphine | about 0.01 mg to about |
| | | 200 mg | | 50 mg |
| 142 | L-DOPA | about 10 mg to about | oxycodone | about 0.01 mg to about |
| | | 200 mg | | 50 mg |
| 143 | L-DOPA | about 10 mg to about | meperidine | about 1 mg to about |
| | | 200 mg | | 250 mg |
| 144 | L-DOPA | about 10 mg to about | codeine | about 1 mg to about |
| | | 200 mg | | 250 mg |

Non-limiting examples of compositions comprising a MAO inhibitor and an opioid agent in accordance with the invention are provided in Table 4.

TABLE 4

| Example | | | | |
|---|---|---|---|---|
| No. | MAO Inhibitor | Dosage | Opioid Agent | Dosage |
| 145 | selegiline | about 1 mg to about | nalbuphine | about 5 mg to about |
| | | 25 mg | | 150 mg |
| 146 | selegiline | about 1 mg to about | nalbuphine | about 5 mg to about 25 mg |
| | | 25 mg | | |
| 147 | selegiline | about 5 mg to about | nalbuphine | about 5 mg to about |
| | | 25 mg | | 150 mg |
| 148 | selegiline | about 5 mg to about | nalbuphine | about 5 mg to about 25 mg |
| | | 25 mg | | |
| 149 | selegiline | about 1 mg | nalbuphine | about 5 mg to about |
| | | | | 150 mg |
| 150 | selegiline | about 5 mg | nalbuphine | about 5 mg to about |
| | | | | 150 mg |
| 151 | selegiline | about 1 mg to about | pentazocine | about 5 mg to about |
| | | 25 mg | | 150 mg |
| 152 | selegiline | about 1 mg to about | butorphanol | about 5 mg to about |
| | | 25 mg | | 150 mg |
| 153 | selegiline | about 1 mg to about | fentanyl | about 0.01 mg to about |
| | | 25 mg | | 25 mg |
| 154 | selegiline | about 1 mg to about | hydrocodone | about 0.01 mg to about |
| | | 25 mg | | 25 mg |
| 155 | selegiline | about 1 mg to about | hydromorphone | about 0.01 mg to about |
| | | 25 mg | | 25 mg |
| 156 | selegiline | about 1 mg to about | morphine | about 0.01 mg to about |
| | | 25 mg | | 50 mg |
| 157 | selegiline | about 1 mg to about | oxycodone | about 0.01 mg to about |
| | | 25 mg | | 50 mg |
| 158 | selegiline | about 1 mg to about | meperidine | about 1 mg to about |
| | | 25 mg | | 250 mg |
| 159 | selegiline | about 1 mg to about | codeine | about 1 mg to about |
| | | 25 mg | | 250 mg |
| 160 | isocarbaxazid | about 10 mg to about | pentazocine | about 5 mg to about |
| | | 200 mg | | 150 mg |
| 161 | isocarbaxazid | about 10 mg to about | butorphanol | about 5 mg to about |
| | | 200 mg | | 150 mg |
| 162 | isocarbaxazid | about 10 mg to about | nalbuphine | about 5 mg to about |
| | | 200 mg | | 150 mg |
| 163 | isocarbaxazid | about 10 mg to about | fentanyl | about 0.01 mg to about |
| | | 200 mg | | 25 mg |
| 164 | isocarbaxazid | about 10 mg to about | hydrocodone | about 0.01 mg to about |
| | | 200 mg | | 25 mg |
| 165 | isocarbaxazid | about 10 mg to about | hydromorphone | about 0.01 mg to about |
| | | 200 mg | | 25 mg |
| 166 | isocarbaxazid | about 10 mg to about | morphine | about 0.01 mg to about |
| | | 200 mg | | 50 mg |
| 167 | isocarbaxazid | about 10 mg to about | oxycodone | about 0.01 mg to about |
| | | 200 mg | | 50 mg |
| 168 | isocarbaxazid | about 10 mg to about | meperidine | about 1 mg to about |
| | | 200 mg | | 250 mg |
| 169 | isocarbaxazid | about 10 mg to about | codeine | about 1 mg to about |
| | | 200 mg | | 250 mg |
| 170 | phenelzine sulfate | about 10 mg to about | pentazocine | about 5 mg to about |
| | | 200 mg | | 150 mg |
| 171 | phenelzine sulfate | about 10 mg to about | butorphanol | about 5 mg to about |
| | | 200 mg | | 150 mg |
| 172 | phenelzine sulfate | about 10 mg to about | nalbuphine | about 5 mg to about |
| | | 200 mg | | 150 mg |
| 173 | phenelzine sulfate | about 10 mg to about | fentanyl | about 0.01 mg to about |
| | | 200 mg | | 25 mg |
| 174 | phenelzine sulfate | about 10 mg to about | hydrocodone | about 0.01 mg to about |
| | | 200 mg | | 25 mg |
| 175 | phenelzine sulfate | about 10 mg to about | hydromorphone | about 0.01 mg to about |
| | | 200 mg | | 25 mg |
| 176 | phenelzine sulfate | about 10 mg to about | morphine | about 0.01 mg to about |
| | | 200 mg | | 50 mg |
| 177 | phenelzine sulfate | about 10 mg to about | oxycodone | about 0.01 mg to about |
| | | 200 mg | | 50 mg |
| 178 | phenelzine sulfate | about 10 mg to about | meperidine | about 1 mg to about |
| | | 200 mg | | 250 mg |
| 179 | phenelzine sulfate | about 10 mg to about | codeine | about 1 mg to about |
| | | 200 mg | | 250 mg |
| 180 | tranylcypromine | about 10 mg to about | pentazocine | about 5 mg to about |
| | sulfate | 200 mg | | 150 mg |
| 181 | tranylcypromine | about 10 mg to about | butorphanol | about 5 mg to about |
| | sulfate | 200 mg | | 150 mg |
| 182 | tranylcypromine | about 10 mg to about | nalbuphine | about 5 mg to about |
| | sulfate | 200 mg | | 150 mg |
| 183 | tranylcypromine | about 10 mg to about | fentanyl | about 0.01 mg to about |
| | sulfate | 200 mg | | 25 mg |
| 184 | tranylcypromine | about 10 mg to about | hydrocodone | about 0.01 mg to about |
| | sulfate | 200 mg | | 25 mg |
| 185 | tranylcypromine | about 10 mg to about | hydromorphone | about 0.01 mg to about |
| | sulfate | 200 mg | | 25 mg |
| 186 | tranylcypromine | about 10 mg to about | morphine | about 0.01 mg to about |
| | sulfate | 200 mg | | 50 mg |
| 187 | tranylcypromine | about 10 mg to about | oxycodone | about 0.01 mg to about |
| | sulfate | 200 mg | | 50 mg |
| 188 | tranylcypromine | about 10 mg to about | meperidine | about 1 mg to about |
| | sulfate | 200 mg | | 250 mg |
| 189 | tranylcypromine | about 10 mg to about | codeine | about 1 mg to about |
| | sulfate | 200 mg | | 250 mg |
| 170 | rasagaline | about 0.1 mg to | pentazocine | about 5 mg to about |
| | | about 1 mg | | 150 mg |
| 171 | rasagaline | about 0.1 mg to | butorphanol | about 5 mg to about |
| | | about 1 mg | | 150 mg |
| 172 | rasagaline | about 0.1 mg to | nalbuphine | about 5 mg to about |
| | | about 1 mg | | 150 mg |
| 173 | rasagaline | about 0.1 mg to | nalbuphine | about 5 mg to about |
| | | about 1 mg | | 150 mg |
| 174 | rasagaline | about 0.5 mg | nalbuphine | about 5 mg to about |
| | | | | 150 mg |
| 175 | rasagaline | about 1 mg | nalbuphine | about 5 mg to about |
| | | | | 150 mg |
| 176 | rasagaline | about 0.1 mg to | fentanyl | about 0.01 mg to about |
| | | about 1 mg | | 25 mg |
| 177 | rasagaline | about 0.1 mg to | hydrocodone | about 0.01 mg to about |
| | | about 1 mg | | 25 mg |
| 178 | rasagaline | about 0.1 mg to | hydromorphone | about 0.01 mg to about |
| | | about 1 mg | | 25 mg |
| 179 | rasagaline | about 0.1 mg to | morphine | about 0.01 mg to about |
| | | about 1 mg | | 50 mg |
| 180 | rasagaline | about 0.1 mg to | oxycodone | about 0.01 mg to about |
| | | about 1 mg | | 50 mg |
| 181 | rasagaline | about 0.1 mg to | meperidine | about 1 mg to about |
| | | about 1 mg | | 250 mg |
| 182 | rasagaline | about 0.1 mg to | codeine | about 1 mg to about |
| | | about 1 mg | | 250 mg |

Non-limiting examples of compositions comprising a dopamine re-uptake inhibitor and an opioid agent in accordance with the invention are provided in Table 5.

TABLE 5

| Example | Dopamine reuptake | | | |
|---|---|---|---|---|
| No. | Inhibitor | Dosage | Opioid Agent | Dosage |
| 183 | amineptine | about 1 mg to about | nalbuphine | about 5 mg to about |
| | | 200 mg | | 150 mg |
| 184 | amineptine | about 1 mg to about | pentazocine | about 5 mg to about |
| | | 200 mg | | 150 mg |
| 185 | amineptine | about 1 mg to about | butorphanol | about 5 mg to about |
| | | 200 mg | | 150 mg |
| 186 | amineptine | about 1 mg to about | fentanyl | about 0.01 mg to |
| | | 200 mg | | about 25 mg |
| 187 | amineptine | about 1 mg to about | hydrocodone | about 0.01 mg to |
| | | 200 mg | | about 25 mg |
| 188 | amineptine | about 1 mg to about | hydromorphone | about 0.01 mg to |
| | | 200 mg | | about 25 mg |
| 189 | amineptine | about 1 mg to about | morphine | about 0.01 mg to |
| | | 200 mg | | about 50 mg |
| 190 | amineptine | about 1 mg to about | oxycodone | about 0.01 mg to |
| | | 200 mg | | about 50 mg |
| 191 | amineptine | about 1 mg to about | meperidine | about 1 mg to about |
| | | 200 mg | | 250 mg |
| 192 | amineptine | about 1 mg to about | codeine | about 1 mg to about |
| | | 200 mg | | 250 mg |
| 193 | bromantane | about 1 mg to about | nalbuphine | about 5 mg to about |
| | | 200 mg | | 150 mg |
| 194 | bromantane | about 1 mg to about | pentazocine | about 5 mg to about |
| | | 200 mg | | 150 mg |
| 195 | bromantane | about 1 mg to about | butorphanol | about 5 mg to about |
| | | 200 mg | | 150 mg |
| 196 | bromantane | about 1 mg to about | fentanyl | about 0.01 mg to |
| | | 200 mg | | about 25 mg |
| 197 | bromantane | about 1 mg to about | hydrocodone | about 0.01 mg to |
| | | 200 mg | | about 25 mg |
| 198 | bromantane | about 1 mg to about | hydromorphone | about 0.01 mg to |
| | | 200 mg | | about 25 mg |
| 199 | bromantane | about 1 mg to about | morphine | about 0.01 mg to |
| | | 200 mg | | about 50 mg |
| 200 | bromantane | about 1 mg to about | oxycodone | about 0.01 mg to |
| | | 200 mg | | about 50 mg |
| 201 | bromantane | about 1 mg to about | meperidine | about 1 mg to about |
| | | 200 mg | | 250 mg |
| 202 | bromantane | about 1 mg to about | codeine | about 1 mg to about |
| | | 200 mg | | 250 mg |
| 203 | dexmethylphenidate | about 1 mg to about | nalbuphine | about 5 mg to about |
| | | 200 mg | | 150 mg |
| 204 | dexmethylphenidate | about 1 mg to about | pentazocine | about 5 mg to about |
| | | 200 mg | | 150 mg |
| 205 | dexmethylphenidate | about 1 mg to about | butorphanol | about 5 mg to about |
| | | 200 mg | | 150 mg |
| 206 | dexmethylphenidate | about 1 mg to about | fentanyl | about 0.01 mg to |
| | | 200 mg | | about 25 mg |
| 207 | dexmethylphenidate | about 1 mg to about | hydrocodone | about 0.01 mg to |
| | | 200 mg | | about 25 mg |
| 208 | dexmethylphenidate | about 1 mg to about | hydromorphone | about 0.01 mg to |
| | | 200 mg | | about 25 mg |
| 209 | dexmethylphenidate | about 1 mg to about | morphine | about 0.01 mg to |
| | | 200 mg | | about 50 mg |
| 210 | dexmethylphenidate | about 1 mg to about | oxycodone | about 0.01 mg to |
| | | 200 mg | | about 50 mg |
| 211 | dexmethylphenidate | about 1 mg to about | meperidine | about 1 mg to about |
| | | 200 mg | | 250 mg |
| 212 | dexmethylphenidate | about 1 mg to about | codeine | about 1 mg to about |
| | | 200 mg | | 250 mg |
| 213 | difemetorex | about 1 mg to about | nalbuphine | about 5 mg to about |
| | | 200 mg | | 150 mg |
| 214 | difemetorex | about 1 mg to about | pentazocine | about 5 mg to about |
| | | 200 mg | | 150 mg |
| 215 | difemetorex | about 1 mg to about | butorphanol | about 5 mg to about |
| | | 200 mg | | 150 mg |
| 216 | difemetorex | about 1 mg to about | fentanyl | about 0.01 mg to |
| | | 200 mg | | about 25 mg |
| 217 | difemetorex | about 1 mg to about | hydrocodone | about 0.01 mg to |
| | | 200 mg | | about 25 mg |
| 218 | difemetorex | about 1 mg to about | hydromorphone | about 0.01 mg to |
| | | 200 mg | | about 25 mg |
| 219 | difemetorex | about 1 mg to about | morphine | about 0.01 mg to |
| | | 200 mg | | about 50 mg |
| 220 | difemetorex | about 1 mg to about | oxycodone | about 0.01 mg to |
| | | 200 mg | | about 50 mg |
| 221 | difemetorex | about 1 mg to about | meperidine | about 1 mg to about |
| | | 200 mg | | 250 mg |
| 222 | difemetorex | about 1 mg to about | codeine | about 1 mg to about |
| | | 200 mg | | 250 mg |
| 223 | fencamfamine | about 1 mg to about | nalbuphine | about 5 mg to about |
| | | 200 mg | | 150 mg |
| 224 | fencamfamine | about 1 mg to about | pentazocine | about 5 mg to about |
| | | 200 mg | | 150 mg |
| 225 | fencamfamine | about 1 mg to about | butorphanol | about 5 mg to about |
| | | 200 mg | | 150 mg |
| 226 | fencamfamine | about 1 mg to about | fentanyl | about 0.01 mg to |
| | | 200 mg | | about 25 mg |
| 227 | fencamfamine | about 1 mg to about | hydrocodone | about 0.01 mg to |
| | | 200 mg | | about 25 mg |
| 228 | fencamfamine | about 1 mg to about | hydromorphone | about 0.01 mg to |
| | | 200 mg | | about 25 mg |
| 229 | fencamfamine | about 1 mg to about | morphine | about 0.01 mg to |
| | | 200 mg | | about 50 mg |
| 230 | fencamfamine | about 1 mg to about | oxycodone | about 0.01 mg to |
| | | 200 mg | | about 50 mg |
| 231 | fencamfamine | about 1 mg to about | meperidine | about 1 mg to about |
| | | 200 mg | | 250 mg |
| 232 | fencamfamine | about 1 mg to about | codeine | about 1 mg to about |
| | | 200 mg | | 250 mg |
| 233 | lefetamine | about 1 mg to about | nalbuphine | about 5 mg to about |
| | | 200 mg | | 150 mg |
| 234 | lefetamine | about 1 mg to about | pentazocine | about 5 mg to about |
| | | 200 mg | | 150 mg |
| 235 | lefetamine | about 1 mg to about | butorphanol | about 5 mg to about |
| | | 200 mg | | 150 mg |
| 236 | lefetamine | about 1 mg to about | fentanyl | about 0.01 mg to |
| | | 200 mg | | about 25 mg |
| 237 | lefetamine | about 1 mg to about | hydrocodone | about 0.01 mg to |
| | | 200 mg | | about 25 mg |
| 238 | lefetamine | about 1 mg to about | hydromorphone | about 0.01 mg to |
| | | 200 mg | | about 25 mg |
| 239 | lefetamine | about 1 mg to about | morphine | about 0.01 mg to |
| | | 200 mg | | about 50 mg |
| 240 | lefetamine | about 1 mg to about | oxycodone | about 0.01 mg to |
| | | 200 mg | | about 50 mg |
| 241 | lefetamine | about 1 mg to about | meperidine | about 1 mg to about |
| | | 200 mg | | 250 mg |
| 242 | lefetamine | about 1 mg to about | codeine | about 1 mg to about |
| | | 200 mg | | 250 mg |
| 243 | levophacetoperane | about 1 mg to about | nalbuphine | about 5 mg to about |
| | | 200 mg | | 150 mg |
| 244 | levophacetoperane | about 1 mg to about | pentazocine | about 5 mg to about |
| | | 200 mg | | 150 mg |
| 245 | levophacetoperane | about 1 mg to about | butorphanol | about 5 mg to about |
| | | 200 mg | | 150 mg |
| 246 | levophacetoperane | about 1 mg to about | fentanyl | about 0.01 mg to |
| | | 200 mg | | about 25 mg |
| 247 | levophacetoperane | about 1 mg to about | hydrocodone | about 0.01 mg to |
| | | 200 mg | | about 25 mg |
| 248 | levophacetoperane | about 1 mg to about | hydromorphone | about 0.01 mg to |
| | | 200 mg | | about 25 mg |
| 249 | levophacetoperane | about 1 mg to about | morphine | about 0.01 mg to |
| | | 200 mg | | about 50 mg |
| 250 | levophacetoperane | about 1 mg to about | oxycodone | about 0.01 mg to |
| | | 200 mg | | about 50 mg |
| 251 | levophacetoperane | about 1 mg to about | meperidine | about 1 mg to about |
| | | 200 mg | | 250 mg |
| 252 | levophacetoperane | about 1 mg to about | codeine | about 1 mg to about |
| | | 200 mg | | 250 mg |
| 253 | medifoxamine | about 1 mg to about | nalbuphine | about 5 mg to about |
| | | 200 mg | | 150 mg |
| 254 | medifoxamine | about 1 mg to about | pentazocine | about 5 mg to about |
| | | 200 mg | | 150 mg |
| 255 | medifoxamine | about 1 mg to about | butorphanol | about 5 mg to about |
| | | 200 mg | | 150 mg |
| 256 | medifoxamine | about 1 mg to about | fentanyl | about 0.01 mg to |
| | | 200 mg | | about 25 mg |
| 257 | medifoxamine | about 1 mg to about | hydrocodone | about 0.01 mg to |
| | | 200 mg | | about 25 mg |
| 258 | medifoxamine | about 1 mg to about | hydromorphone | about 0.01 mg to |
| | | 200 mg | | about 25 mg |
| 259 | medifoxamine | about 1 mg to about | morphine | about 0.01 mg to |
| | | 200 mg | | about 50 mg |
| 260 | medifoxamine | about 1 mg to about | oxycodone | about 0.01 mg to |
| | | 200 mg | | about 50 mg |
| 261 | medifoxamine | about 1 mg to about | meperidine | about 1 mg to about |
| | | 200 mg | | 250 mg |
| 262 | medifoxamine | about 1 mg to about | codeine | about 1 mg to about |
| | | 200 mg | | 250 mg |
| 263 | mesocarb | about 1 mg to about | nalbuphine | about 5 mg to about |
| | | 200 mg | | 150 mg |
| 264 | mesocarb | about 1 mg to about | pentazocine | about 5 mg to about |
| | | 200 mg | | 150 mg |
| 265 | mesocarb | about 1 mg to about | butorphanol | about 5 mg to about |
| | | 200 mg | | 150 mg |
| 266 | mesocarb | about 1 mg to about | fentanyl | about 0.01 mg to |
| | | 200 mg | | about 25 mg |
| 267 | mesocarb | about 1 mg to about | hydrocodone | about 0.01 mg to |
| | | 200 mg | | about 25 mg |
| 268 | mesocarb | about 1 mg to about | hydromorphone | about 0.01 mg to |
| | | 200 mg | | about 25 mg |
| 269 | mesocarb | about 1 mg to about | morphine | about 0.01 mg to |
| | | 200 mg | | about 50 mg |
| 270 | mesocarb | about 1 mg to about | oxycodone | about 0.01 mg to |
| | | 200 mg | | about 50 mg |
| 271 | mesocarb | about 1 mg to about | meperidine | about 1 mg to about |
| | | 200 mg | | 250 mg |
| 272 | mesocarb | about 1 mg to about | codeine | about 1 mg to about |
| | | 200 mg | | 250 mg |
| 273 | methylphenidate | about 1 mg to about | nalbuphine | about 5 mg to about |
| | | 200 mg | | 150 mg |
| 274 | methylphenidate | about 1 mg to about | pentazocine | about 5 mg to about |
| | | 200 mg | | 150 mg |
| 275 | methylphenidate | about 1 mg to about | butorphanol | about 5 mg to about |
| | | 200 mg | | 150 mg |
| 276 | methylphenidate | about 1 mg to about | fentanyl | about 0.01 mg to |
| | | 200 mg | | about 25 mg |
| 277 | methylphenidate | about 1 mg to about | hydrocodone | about 0.01 mg to |
| | | 200 mg | | about 25 mg |
| 278 | methylphenidate | about 1 mg to about | hydromorphone | about 0.01 mg to |
| | | 200 mg | | about 25 mg |
| 279 | methylphenidate | about 1 mg to about | morphine | about 0.01 mg to |
| | | 200 mg | | about 50 mg |
| 280 | methylphenidate | about 1 mg to about | oxycodone | about 0.01 mg to |
| | | 200 mg | | about 50 mg |
| 281 | methylphenidate | about 1 mg to about | meperidine | about 1 mg to about |
| | | 200 mg | | 250 mg |
| 282 | methylphenidate | about 1 mg to about | codeine | about 1 mg to about |
| | | 200 mg | | 250 mg |
| 283 | nomifensine | about 1 mg to about | nalbuphine | about 5 mg to about |
| | | 200 mg | | 150 mg |
| 284 | nomifensine | about 1 mg to about | pentazocine | about 5 mg to about |
| | | 200 mg | | 150 mg |
| 285 | nomifensine | about 1 mg to about | butorphanol | about 5 mg to about |
| | | 200 mg | | 150 mg |
| 286 | nomifensine | about 1 mg to about | fentanyl | about 0.01 mg to |
| | | 200 mg | | about 25 mg |
| 287 | nomifensine | about 1 mg to about | hydrocodone | about 0.01 mg to |
| | | 200 mg | | about 25 mg |
| 288 | nomifensine | about 1 mg to about | hydromorphone | about 0.01 mg to |
| | | 200 mg | | about 25 mg |
| 289 | nomifensine | about 1 mg to about | morphine | about 0.01 mg to |
| | | 200 mg | | about 50 mg |
| 290 | nomifensine | about 1 mg to about | oxycodone | about 0.01 mg to |
| | | 200 mg | | about 50 mg |
| 291 | nomifensine | about 1 mg to about | meperidine | about 1 mg to about |
| | | 200 mg | | 250 mg |
| 292 | nomifensine | about 1 mg to about | codeine | about 1 mg to about |
| | | 200 mg | | 250 mg |
| 293 | pipradrol | about 1 mg to about | nalbuphine | about 5 mg to about |
| | | 200 mg | | 150 mg |
| 294 | pipradrol | about 1 mg to about | pentazocine | about 5 mg to about |
| | | 200 mg | | 150 mg |
| 295 | pipradrol | about 1 mg to about | butorphanol | about 5 mg to about |
| | | 200 mg | | 150 mg |
| 296 | pipradrol | about 1 mg to about | fentanyl | about 0.01 mg to |
| | | 200 mg | | about 25 mg |
| 297 | pipradrol | about 1 mg to about | hydrocodone | about 0.01 mg to |
| | | 200 mg | | about 25 mg |
| 298 | pipradrol | about 1 mg to about | hydromorphone | about 0.01 mg to |
| | | 200 mg | | about 25 mg |
| 299 | pipradrol | about 1 mg to about | morphine | about 0.01 mg to |
| | | 200 mg | | about 50 mg |
| 300 | pipradrol | about 1 mg to about | oxycodone | about 0.01 mg to |
| | | 200 mg | | about 50 mg |
| 301 | pipradrol | about 1 mg to about | meperidine | about 1 mg to about |
| | | 200 mg | | 250 mg |
| 302 | pipradrol | about 1 mg to about | codeine | about 1 mg to about |
| | | 200 mg | | 250 mg |
| 303 | prolintane | about 1 mg to about | nalbuphine | about 5 mg to about |
| | | 200 mg | | 150 mg |
| 304 | prolintane | about 1 mg to about | pentazocine | about 5 mg to about |
| | | 200 mg | | 150 mg |
| 305 | prolintane | about 1 mg to about | butorphanol | about 5 mg to about |
| | | 200 mg | | 150 mg |
| 306 | prolintane | about 1 mg to about | fentanyl | about 0.01 mg to |
| | | 200 mg | | about 25 mg |
| 307 | prolintane | about 1 mg to about | hydrocodone | about 0.01 mg to |
| | | 200 mg | | about 25 mg |
| 308 | prolintane | about 1 mg to about | hydromorphone | about 0.01 mg to |
| | | 200 mg | | about 25 mg |
| 309 | prolintane | about 1 mg to about | morphine | about 0.01 mg to |
| | | 200 mg | | about 50 mg |
| 310 | prolintane | about 1 mg to about | oxycodone | about 0.01 mg to |
| | | 200 mg | | about 50 mg |
| 311 | prolintane | about 1 mg to about | meperidine | about 1 mg to about |
| | | 200 mg | | 250 mg |
| 312 | prolintane | about 1 mg to about | codeine | about 1 mg to about |
| | | 200 mg | | 250 mg |
| 313 | pyrovalerone | about 1 mg to about | nalbuphine | about 5 mg to about |
| | | 200 mg | | 150 mg |
| 314 | pyrovalerone | about 1 mg to about | pentazocine | about 5 mg to about |
| | | 200 mg | | 150 mg |
| 315 | pyrovalerone | about 1 mg to about | butorphanol | about 5 mg to about |
| | | 200 mg | | 150 mg |
| 316 | pyrovalerone | about 1 mg to about | fentanyl | about 0.01 mg to |
| | | 200 mg | | about 25 mg |
| 317 | pyrovalerone | about 1 mg to about | hydrocodone | about 0.01 mg to |
| | | 200 mg | | about 25 mg |
| 318 | pyrovalerone | about 1 mg to about | hydromorphone | about 0.01 mg to |
| | | 200 mg | | about 25 mg |
| 319 | pyrovalerone | about 1 mg to about | morphine | about 0.01 mg to |
| | | 200 mg | | about 50 mg |
| 320 | pyrovalerone | about 1 mg to about | oxycodone | about 0.01 mg to |
| | | 200 mg | | about 50 mg |
| 321 | pyrovalerone | about 1 mg to about | meperidine | about 1 mg to about |
| | | 200 mg | | 250 mg |
| 322 | pyrovalerone | about 1 mg to about | codeine | about 1 mg to about |
| | | 200 mg | | 250 mg |

In certain embodiments the weight ratio of dopaminergic agent to opioid agent is in the range of: 50000:1 to 1:50000, 20000:1 to 1:20000, 10000:1 to 1:10000, 5000:1 to 1:5000, 2500:1 to 1:2500, 2000:1 to 1:2000, 1500:1 to 1:1500, 1000:1 to 1:1000, 750:1 to 1:750, 500:1 to 1:500, 250:1 to 1:250, 100:1 to 1:100, 75:1 to 1:75, 50:1 to 1:50, 25:1 to 1:25, 20:1 to 1:20, 2:1 to 1:2:4:1 to 1:2, 1:1 to 1:50000, 1:1 to 1:20000, 1:1 to 1:10000, 1:1 to 1:7500, 1:1 to 1:5000, 1:1 to 1:2500, 1:1 to 1:1000, 1:1 to 1:750, 1:1 to 1:500, 1:1 to 1:250, 1:1 to 1:100, 1:1 to 1:75, 1:1 to 1:50, 1:1 to 1:25, 1:1 to 1:20, 1:1 to 1:10, 1:1 to 1:5, 1:1 to 50000:1, 1:1 to 20000:1, 1:1 to 10000:1, 1:1 to 7500:1, 1:1 to 5000:1, 1:1 to 2500:1, 1:1 to 1000:1, 1:1 to 750:1, 1:1 to 500:1, 1:1 to 250:1, 1:1 to 100:1, 1:1 to 75:1, 1:1 to 50:1, 1:1 to 25, 1:1 to 20:1, 1:1 to 10:1, 1:1 to 5:1.

In an embodiment, a dopaminergic agent and an opioid agent can be formulated separately or together in various administration vehicles, including, but not limited to, tablet, orally disintegrating tablet, capsule, syrup, suppository, transdermal delivery system (e.g. skin patch), inhalable powder, inhalable aerosol, sublingual spray, intranasal spray and intranasal aerosol. In some embodiments a dopaminergic agent and/or an opioid agent may be administered via oral, rectal, buccal, intranasal or transdermal routes, by intra-arterial injection, intravenously, intraperitoneally, parenterally, intramuscularly, subcutaneously, sublingually, orally, topically, or as an inhalant. The major advantage of utilizing such a non-injectable form is to improve the quality of life and compliance for patients requiring repeated or chronic administration. In some embodiments a dopaminergic agent and an opioid agent may be administered concomitantly in the same formulation and administration vehicle. In some embodiments the dopaminergic agent may be administered in separate formulations but via the same administration vehicle (e.g. two tablets, one comprising a dopaminergic agent and one comprising an opioid agent). In some embodiments a dopaminergic agent and an opioid agent may be administered by different administration vehicles. For example, in some embodiments one of the dopaminergic agent and the opioid agent may be administered in an injectable form and the other may be administered in a non-injectable form.

In some embodiments, the present invention includes one or more additional constituents having pharmacological activity. Such additional constituents include mu-opioid receptor antagonists/kappa-opioid receptor agonists, components for suppressing peripheral metabolism (e.g., carbidopa and benserazide), and/or other additional active ingredients such as, for instance, painkillers, antibacterial, antiviral or antifungal agents, vitamins, immune system fortifiers, homeopathic agents, antihypertensive medication, nootropics, any combinations thereof, and so forth.

Other Constituents

In some embodiments of the invention, inactive ingredients or other additives may be included. Such inactive ingredients can be used for bulk, drug release properties, as a carrier, for facilitating digestion, and for other purposes, as known in the art. In one example, a formulation includes Nalbuphine, L-DOPA, a release modifying component, and optional additives.

In some embodiments a composition includes a release-modifying component. Preferably, the one or more materials prolong the release of opioid (e.g. nalbuphine) from the composition. Examples of release-modifying materials include carbomers, carboxymethylcellulose, hydroxypropylmethylcellulose, biodegradable polymers, as well as any combination thereof. Carbomers, e.g., Carbopole resins are compounds that are carboxyacrylic or carboxyvinyl polymers.

Additives such as, for example, lactose, microcrystalline cellulose, colloidal silica, lubricants, acid stabilizers, disintegrants and many others also can be included.

Some additives that can be employed include, but are not limited to ammonio-methacrylate copolymers, NF, fumaric acid, NF, povidone, USP, sodium lauryl sulfate, NF, sugar starch, NF, and talc, USP, gelatin, titanium dioxide, lactose, hydroxypropyl methylcellulose, colloidal silicon dioxide, stearic acid, lactic acid, citric acid, vitamin E, EDTA, butylated hydroxyanisole, propylparaben, methylparaben, sodium benzoate, potassium benzoate, benzalkonium chloride, benzoic acid, sorbic acid, PEG 400, carrageenan products (such as Viscarin 328, Gelcarin 812, and Seaspen), microcrystalline cellulose (MCC), colloid silicon oxide (e.g., Aerosil) and others.

In some embodiments a composition may include a lubricant. Examples of lubricants include magnesium stearate, calcium stearate, talcum, their mixtures and others, as known in the art. It is preferably to use magnesium stearate in the quantity of 0.2-1.5% and talcum in the quantity of 0.8-3.0%.

In some embodiments a composition may include lactose. Lactose is a neutral filler, providing optimal rheological properties of the granulated material and tablet mass in the manufacture of the tablet. Lactose having particle size of 70-200 microns, are preferred. Also preferred are spherical or nearly spherical lactose particles.

In a specific, non-limiting example, a non-injectable formulation is a capsule containing 5-150 mg (e.g. 5, 10, 60, 90, or 150 mg) of nalbuphine, 20-100 mg of L-DOPA, and one or more of the inactive ingredients such as ammonio-methacrylate copolymers, NF, fumaric acid, NF, povidone, USP, sodium lauryl sulfate, NF, sugar starch spheres, NF, and talc, USP, (and their suitable analogues). The capsule shell can contain ink, gelatin, titanium dioxide, (and their suitable analogues).

In another specific, non-limiting example, a non-injectable formulation is a tablet including 5-150 mg (e.g. 5, 10, 60, 90, or 150 mg) of nalbuphine, 10-200 mg of L-DOPA, and one or more of the inactive ingredients such as, lactose, hydroxypropyl methylcellulose, colloidal silicon dioxide, and/or stearic acid. Known analogues of the inactive components also can be used.

In another specific, non-limiting example a tablet form includes by weight: pramipexole 0.1-4.5 mg; nalbuphine chloride 5-150 mg. A tablet may also contain hypromellose, corn starch, carbomer homopolymer, colloidal silicon dioxide, magnesium stearate, or a combination thereof.

Another example of the formulation in tablet form includes by weight: entacapone 10-200 mg; nalbuphine chloride: 5-150 mg. A tablet may also contain starch or hydroxypropyl cellulose, pregelatinized starch, crospovidone, microcrystalline cellulose, magnesium stearate, or a combination thereof.

Another example of the formulation in tablet form includes by weight: L-DOPA 10-200 mg; carbidopa 5-25 mg; nalbuphine chloride: 5-150 mg. A tablet may also contain starch or hydroxypropyl cellulose, pregelatinized starch, crospovidone, microcrystalline cellulose, magnesium stearate, or a combination thereof.

Yet another example of the formulation in tablet form includes by weight: L-DOPA 25-50 mg; carbidopa 6-12.5 mg; nalbuphine chloride: 5-150 mg. A tablet may also contain starch or hydroxypropyl cellulose, pregelatinized starch, crospovidone, microcrystalline cellulose, magnesium stearate, or a combination thereof.

Another example of the formulation in tablet form includes by weight: L-DOPA 10-200 mg; carbidopa 5-25 mg; entacapone 10-200 mg; nalbuphine chloride: 5-150 mg. A tablet may also contain starch or hydroxypropyl cellulose, pregelatinized starch, crospovidone, microcrystalline cellulose, magnesium stearate, or a combination thereof.

Yet another example of the formulation in tablet form includes by weight: L-DOPA 25-50 mg; carbidopa 6-12.5 mg; entacapone 10-200 mg; nalbuphine chloride: 5-150 mg. A tablet may also contain starch or hydroxypropyl cellulose, pregelatinized starch, crospovidone, microcrystalline cellulose, magnesium stearate, or a combination thereof.

Without wishing to be held by a specific mechanism of action, it is believed that compounds employed in the present formulation can form a protective matrix around active ingredients and modify its release kinetics from the formulation.

A tablet or caplet described above can be formulated at a desired dosage, for example, it can contain 5-150 mg of nalbuphine chloride and 0.1-4.5 mg pramipexole or 10-200 mg of COMT inhibitor or 10-200 mg of L-DOPA.

The non-injectable formulations disclosed herein can be prepared by combining one or more agonist-antagonists with any other active or inactive ingredients. The process is not limited to any particular order of adding ingredients. One or more ingredients can be added simultaneously and sequential additions also can be carried out. Laboratory, pilot plant and commercial operations can be employed. Mixing, spray drying, emulsifying, purifying, compounding, and many other additional steps known in the fields of drug synthesis and manufacture also can be used to produce the non-injectable formulation.

Methods to Reduce Opioid Sedation

In an embodiment, the present invention is a method of reducing, preventing, or eliminating opiate adverse effects such as sedation by administering separately, or simultaneously as a composition, comprising a dopaminergic agent and an opioid agent. In an embodiment the dopaminergic agent is one or more of (a) a dopamine agonist, (b) L-DOPA, (c) an MAO inhibitor, (d) a COMT inhibitor, or (e) a dopamine re-uptake inhibitor.

In an embodiment, the present invention is a method of reducing, preventing, or eliminating opiate adverse effects such as sedation by administering separately or simultaneously as a composition comprising a dopamine agonist and an opioid agent. In an embodiment a dopamine agonist is administered at a dosage range of about 0.1 mg to about 25 mg, and an opioid agent is administered at a dosage range of about 0.01 mg to about 150 mg. In some embodiments the dopamine agonist is selected from the group consisting of pramipexole, ropinirole, rotigotine, apomorphine, piribedil, cabergoline, lisuride, and derivatives, prodrugs, esters, and salts thereof. In some embodiments the opioid agent is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof. In one embodiment the dopamine agonist is pramipexole and the opioid agent is nalbuphine.

In an embodiment, the present invention is a method of reducing, preventing, or eliminating opiate adverse effects such as sedation by administering separately or simultaneously as a composition comprising a COMT inhibitor and an opioid agent. In an embodiment a COMT inhibitor is administered at a dosage range of about 10 mg to about 200 mg, and an opioid agent is administered at a dosage range of about 0.01 mg to about 150 mg. In some embodiments the COMT inhibitor is selected from the group consisting of entacapone, tolcapone, nitecapone, opicapone, and derivatives, prodrugs, esters, and salts thereof. In some embodiments the opioid agent is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof. In one embodiment the COMT inhibitor is entacapone and the opioid agent is nalbuphine.

In an embodiment, the present invention is a method of reducing, preventing, or eliminating opiate adverse effects such as sedation by administering separately or simultaneously as a composition comprising L-DOPA and an opioid agent. In an embodiment L-DOPA is administered at a dosage range of about 10 mg to about 200 mg, and an opioid agent is administered at a dosage range of about 5 mg to about 150 mg. In some embodiments the opioid agent is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof. In one embodiment the opioid agent is nalbuphine.

In an embodiment, the present invention is a method of reducing, preventing, or eliminating opiate adverse effects such as sedation by administering separately or simultaneously as a composition comprising a MAO inhibitor and an opioid agent. In an embodiment a MAO inhibitor is administered at a dosage range of about 0.5 mg to about 10 mg, and an opioid agent is administered at a dosage range of about 0.01 mg to about 150 mg. In some embodiments the MAO inhibitor is selected from the group consisting of isocarboxazid, nialamide, phenelzine, hydracarbazine, tranylcypromie, moclobemide, pirlindole, toloxatone, rasagiline, selegiline, linezolid, and derivatives, prodrugs, esters, and salts thereof. In some embodiments the opioid agent is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof. In one embodiment the MAO inhibitor is an MAO B inhibitor. In one embodiment the MAO inhibitor is selegiline and the opioid agent is nalbuphine. In another embodiment the MAO inhibitor is rasagiline and the opioid agent is nalbuphine.

In some embodiments the opioid agent is administered in a dosage range of about 0.01 mg to about 100 mg, about 0.01 mg to about 10 mg, about 0.01 mg to about 1 mg, about 0.025 mg to about 0.5 mg, about 0.025 mg to about 0.25 mg, about 0.05 mg to about 0.1 mg, about 10 mg to about 100 mg, about 20 mg to about 80 mg, about 25 mg to about 75 mg, about 40 mg to about 60 mg, about 5 mg to about 25 mg, about 25 mg to about 50 mg, about 50 mg to about 75 mg, about 75 mg to about 100 mg, about 100 mg to about 125 mg, or about 125 mg to about 150 mg. In some embodiments the opioid agent is administered at a dose of about 0.01 mg, about 0.025 mg, about 0.05 mg, about 0.075 mg, about 0.1 mg, about 0.15 mg, about 0.2 mg, about 0.25 mg, about 0.3 mg, about 0.35 mg, about 0.4 mg, about 0.45 mg, about 0.5 mg, about 0.6 mg, about 0.7 mg, about 0.75 mg, about 0.8 mg, about 0.9 mg, about 1 mg, about 2 mg, about 2.5 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In one embodiment the dopaminergic agent is administered to a subject at a dose of about 0.1 mg to about 200 mg and the opioid agent is administered to the subject at a dose of about 0.01 mg to about 150 mg.

Additionally, certain embodiments of the present invention includes a method of reducing or eliminating opiate adverse effects such as sedation comprising administering to a subject an injectable form of mu-opioid receptor antagonist/kappa-opioid receptor agonist along with L-DOPA or dopamine agonist when warranted by subject's medical condition.

In one embodiment, the present invention is a method of reducing or eliminating opiate adverse effects such as sedation by administering L-DOPA to a subject as a non-injectable composition comprising: (i) pharmaceutically acceptable oral formulation comprising COMT inhibitor in an amount of at least 10 mg and (ii) free-base nalbuphine, its prodrug or pharmaceutically acceptable salt of nalbuphine in an amount of at least 1 milligram and (iii) an inactive component; and wherein the formulation is in tablet or capsule or oral liquid form.

In another embodiment, the present invention is a method of reducing or eliminating opiate adverse effects such as sedation by administering L-DOPA to a subject a non-injectable composition comprising: (i) pharmaceutically acceptable oral formulation comprising L-DOPA in an amount of at least 10 mg with or without components suppressing peripheral metabolism such as carbidopa and benserazide and (ii) free-base nalbuphine, its prodrug or pharmaceutically acceptable salt of nalbuphine in an amount of at least 1 milligram and (iii) an inactive component; and wherein the formulation is in tablet or capsule or oral liquid form.

In a further embodiment, the present invention pertains to selecting a subject with low dopamine levels by administering nalbuphine and measuring sedation as means of early detection, assessment of risk factors or diagnostics of a relevant condition or a disease.

### Example 1

### Drug Effects on the Nervous System (Sedation) in Tests of Nalbuphine Monotherapy

Parkinsonian non-human primates (n=6) were injected (s.c.) with Nalbuphine HCl (NB, dissolved in saline) and evaluated for changes in parkinsonian motor disability (MDS) and drug effects on the nervous system (DENS). Evaluations were performed immediately before injection (baseline), at 30 minutes post-injection and again every 20 minutes thereafter for 110 minutes or until effects were no longer observable. Five NB doses (0.0, 0.016, 0.05, 0.16, 0.50 mg/kg) were tested in each animal with each dose tested 3 times in all animals.

| | NB DOSE (mg/kg) | | | | |
|---|---|---|---|---|---|
| Sedation Items: | 0.00 | 0.016 | 0.05 | 0.16 | 0.5 |
| Attentiveness | 0.33 ± 0.25 | 2.00 ± 0.73 | 2.75 ± 1.07 | 3.17 ± 1.51 | 5.17 ± 1.34 |
| Reactivity | 0.19 ± 0.12 | 0.67 ± 0.36 | 1.58 ± 0.85 | 4.1 ± 1.60 | 6.65 ± 2.15 |
| Eye Movement | 0.02 ± 0.02 | 0.17 ± 0.17 | 0.54 ± 0.25 | 2.44 ± 0.94 | 3.29 ± 1.10 |
| Mouth* | 0.21 ± 0.16 | 0.13 ± 0.09 | 0.17 ± 0.11 | 0.00 | 1.17 ± 1.07 |
| Appetite^{#} | 0.29 ± 0.29 | 0.13 ± 0.13 | 0.56 ± 0.26 | 1.48 ± 1.03 | 4.79 ± 0.78 |
| Total | 1.04 | 3.08 | 5.61 | 11.19 | 21.06 |

Itemized Sedation scores (see Uthayathas et al., “Assessment of adverse effects of neurotropic drugs in monkeys with the ‘drug effects on the nervous system’ (DENS) scale,” J Neurosci Methods. 2013 Apr. 30; 215(1):97-102, for complete description of the DENS scale, incorporated by reference herein in its entirety). The items in this scale are given in the table with the exception of “Involuntary Movements”, “Bowel” and “Bladder”, which were omitted because they were not scored with this scale. Scores are total values from adding all intervals. Higher scores reflect increased sedative effects. All items were evaluated based on comparison with the baseline parkinsonian state. *Mouth (i.e. excessive salivation—drooling) was only scored when it was noticeably increased compared to the baseline parkinsonian state. ^{#}Appetite was only scored when it was considered markedly reduced based on the animal's interest in taking treats compared to the baseline parkinsonian state. Values represent the means±SEM of all animals (n=6).

### Example 2

### Drug Effects on the Nervous System (Sedation) in Tests of Nalbuphine Co-Administration with LDOPA

Animals (n=6) were injected (s.c.) with NB immediately prior to receiving L-dopa (LD; levodopa methyl ester plus 25% benserazide, dissolved in saline and given s.c.). The LD dose was determined individually for each animal based on ‘on’ response and consistency of dyskinesia. Animals were evaluated for changes in parkinsonian motor disability scores (MDS), drug effects on the nervous system (Sedation) and severity of dyskinesias. Evaluations were performed immediately before injections (baseline), at 30 minutes post-injections and again every 20 minutes thereafter until scores returned to 50-100% of baseline. Each of 5 NB doses (0.0, 0.03, 0.06, 0.13, 0.25 mg/kg) was tested in combination with LD (the same dose for all tests) in each animal with each test repeated 3 times.

| | NB DOSE (mg/kg) with L-DOPA | | | | |
|---|---|---|---|---|---|
| Sedation Items: | 0.00 | 0.003 | 0.0625 | 0.125 | 0.25 |
| Attentiveness | 0.00 | 0.00 | 0.00 | 0.00 | 0.00 |
| Reactivity | 0.00 | 0.00 | 0.00 | 0.00 | 0.00 |
| Eye Movement | 0.00 | 0.00 | 0.00 | 0.00 | 0.00 |
| Mouth* | 0.00 | 0.00 | 0.00 | 0.00 | 0.00 |
| Appetite^{#} | 0.00 | 0.00 | 0.00 | 0.00 | 0.00 |
| Total | 0.00 | 0.00 | 0.00 | 0.00 | 0.00 |

Itemized Sedation scores (see Uthayathas 2013 for complete description of the DENS scale). The items in this scale are given in the table with the exception of “Involuntary Movements”, “Bowel” and “Bladder”, which were omitted because they were not scored with DENS scale. Scores are total values from adding all intervals. Higher scores reflect increased sedative effects. All items were evaluated based on comparison with the baseline parkinsonian state. *Mouth (i.e. excessive salivation—drooling) was only scored when it was noticeably increased compared to the baseline parkinsonian state. ^{#}Appetite was only scored when it was considered markedly reduced based on the animal's interest in taking treats compared to the baseline parkinsonian state. Values represent the mean±SEM of all animals (n=6).

### Example 4

### Compositions Useful to Reduce Adverse Effects Such as Sedation of Nalbuphine

Entacapone 5-40%; Nalbuphine chloride: 5-20%. Tablet contains release modifying component: 10-35%; Colloid silicon oxide e.g., Aerosil: 0.2-3%; Microcrystalline cellulose: 5-20%; Povidone: 1-5%; Lubricants: 0.3-5%; Lactose: the rest. Additionally the tablet could be coated with Opadry YS-1-7027 (white) and ACRYL-EZE (white) with antifoaming emulsion in the following ratio: Opadry YS-1-7027 (white)—16-21.5% ACRYL-EZE (white)—78-83.5%. Antifoaming emulsion—the remaining balance.

Sustained release: Tablet L-DOPA 5-22%; Nalbuphine chloride: 2.5-22%. Tablet contains release modifying component: 10-35%; Colloid silicon oxide e.g., Aerosil: 0.2-3%; Microcrystalline cellulose: 5-20%; Povidone: 1-5%; Lubricants: 0.3-5%; Lactose: the rest. Additionally the tablet could be coated with Opadry YS-1-7027 (white) and ACRYL-EZE (white) with antifoaming emulsion in the following ratio: Opadry YS-1-7027 (white)—16-21.5% ACRYL-EZE (white)—78-83.5%. Antifoaming emulsion—the remaining balance.

Sustained release: Tablet L-DOPA 5-22%; Carbidopa 1.25-5.5%; Nalbuphine chloride: 3-22%. Tablet contains release modifying component: 10-35%; Colloid silicon oxide e.g., Aerosil: 0.2-3%; Microcrystalline cellulose: 5-20%; Povidone: 1-5%; Lubricants: 0.3-5%; Lactose: the rest. Additionally the tablet could be coated with Opadry YS-1-7027 (white) and ACRYL-EZE (white) with antifoaming emulsion in the following ratio: Opadry YS-1-7027 (white)—16-21.5% ACRYL-EZE (white)—78-83.5%. Antifoaming emulsion—the remaining balance.

The foregoing constructive examples and descriptions of the preferred embodiments should be interpreted as illustrating, rather than as limiting the present invention as defined by the claims. All variations and combinations of the features above are intended to be within the scope of the following claims.

What is claimed is:

1. A method of reducing or preventing one or more symptoms of sedation comprising administering a dopaminergic agent and an opioid to a subject.

2. The method of claim 1, wherein the one or more symptoms of sedation are selected from decreased attentiveness, diminished reactivity, drowsiness, and combinations thereof.

3. The method of claim 1, wherein the dopaminergic agent and opioid are administered simultaneously.

4. The method of claim 1, wherein the dopaminergic agent and opioid are administered sequentially.

5. The method of claim 1, wherein the dopaminergic agent is L-DOPA or a derivative, prodrug, ester, or pharmaceutically acceptable salt thereof.

6. The method of claim 1, wherein the dopaminergic agent is a dopamine agonist or a derivative, prodrug, ester, or pharmaceutically acceptable salt thereof.

7. The method of claim 1, wherein the dopaminergic agent is a COMT inhibitor or a derivative, prodrug, ester, or pharmaceutically acceptable salt thereof.

8. The method of claim 1, wherein the dopaminergic agent is a MAO inhibitor or a derivative, prodrug, ester, or pharmaceutically acceptable salt thereof.

9. The method of claim 1, wherein the dopaminergic agent is a dopamine re-uptake inhibitor or a derivative, prodrug, ester, or pharmaceutically acceptable salt thereof.

10. The method of claim 1, wherein the opioid is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof.

11. The method of claim 10, wherein the dopaminergic agent is selected from L-DOPA, selegiline, rasagiline, tolcapone, entacapone, derivatives, prodrugs, esters, and salts thereof, and the opioid is selected from nalbuphine, pentazocine, butorphanol, derivatives, prodrugs, esters, and salts thereof.

12. The method of claim 1, wherein the dopaminergic agent is L-DOPA and the opioid is nalbuphine.

13. The method of claim 1, wherein the dopaminergic agent and opioid are administered for treating pain, dermatological disorder, pruritis, addiction or dystonia.

14. A composition comprising a dopaminergic agent and an opioid, wherein the dopaminergic agent and opioid are present in an amount sufficient to provide the benefit of the opioid and diminish one or more symptoms of decreased attentiveness, diminished reactivity, drowsiness in a patient compared to the same amount of opioid agent alone.

15. The composition of claim 14, wherein the dopaminergic agent is L-DOPA, pramipexole, ropinirole, rotigotine, selegiline, rasagiline, tolcapone, entacapone, or a derivative, prodrug, ester, or salt thereof.

16. The composition of claim 14, wherein the opioid is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof.

17. The composition of claim 16, wherein the opioid is nalbuphine, pentazocine, butorphanol, or a derivative, prodrug, ester, or salt thereof.

18. A composition comprising a dopaminergic agent and an opioid for treating dermatological disorder, pruritis, addiction, dystonia, or a combination thereof, wherein the dopaminergic agent and opioid are present in an amount sufficient to provide the benefit of the opioid and to diminish one or more symptoms of decreased attentiveness, diminished reactivity, drowsiness in a patient compared to the same amount of opioid alone.

19. The composition of claim 18, wherein the dopaminergic agent is L-DOPA, pramipexole, ropinirole, rotigotine, rasaligine, selegiline, tolcapone, entacapone, or a derivative, prodrug, or salt thereof.

20. The composition of claim 19, wherein the opioid is selected from the group consisting of fentanyl, hydrocodone, hydromorphine, morphine, oxycodone, diacetylmorphine, methadone, alfentanil, buprenorphine, carfentanil, codeine, dezocine, dihydrocodeine, dihydromorphine, diphenoxylate, diprenorphine, etorphine, β-hydroxy-3-methylfentanyl, levomethadryl, levorphanol, lofentanil, meperidine, nalmefene, oxymorphone, pethidine, propoxyphene, sufentanil, tilidine, nalbuphine, pentazocine, butorphanol, derivatives, prodrugs, esters, and salts thereof.
